(19) United States
(12) Patent Application Publication
(10) Pub. No.: US 2009/0005319 A1
(43) Pub. Date: Jan. 1, 2009

(54) TOPICAL COMPOSITIONS FOR DELAYING EJACULATION AND METHODS OF USING THE SAME

(76) Inventors: Frank V. Barone, JR., Staten Island, NY (US); Christopher Jacobsen, Staten Island, NY (US); Kirill Chumenko, Staten Island, NY (US)

Correspondence Address: GREENBERG TRAURIG, LLP, 1200 SEVENTEENTH STREET, SUITE 2400, DENVER, CO, 80202, US

(21) Appl. No.: 12/166,100

(22) Filed: Jul. 1, 2008

Related U.S. Application Data
(60) Provisional application No. 60/947,430, filed on Jul. 1, 2007.

(51) Int. Cl. A61K 38/08; A61K 38/05; A61P 15/12

(52) U.S. Cl. 514/17; 514/19

(57) ABSTRACT

A method, composition, and kit for topical application of a composition to a male penis to delay premature ejaculation during intercourse without adversely affecting response in a female partner are disclosed. The method includes applying to the penis a topical composition immediately prior to intercourse. The composition includes an effective amount of a vasodilator agent and a desensitizing agent such as an acetyl dipeptide-1 cetyl ester.

### CROSS REFERENCE TO RELATED APPLICATIONS

This application claims the benefit of priority of U.S. Provisional Patent Application Ser. No. 60/947,430, filed Jul. 1, 2007, the content of which is hereby incorporated by reference in its entirety.

### FIELD OF THE DISCLOSURE

The disclosure relates to topical compositions and more particularly to a topical composition which can be applied prior to sexual activity for the purpose of delaying the onset of ejaculation in a male person.

### BACKGROUND OF THE DISCLOSURE

Rapid and premature ejaculation are debilitating yet common sexual dysfunctions, and have been estimated to affect at least 30 to 40 percent of men at some point in their lives (Derogatis (1980) Med. Aspects Hum. Sexuality 14:1168-76; Frank et al. (1978) New Engl. J. Med. 299:111-115; Schein et al. (1988) Fam. Pract. Res. J. 7(3):122-134). Hypersensitivity of sexual stimulation is caused by a disorder in the complex interaction between the peripheral nervous system and the central nervous system. An organic origin for hypersensitivity of the glans penis in patients with premature ejaculation has been shown to cause hypersensitivity and hyperexcitability of the glans penis giving rise to uncontrolled ejaculation. Hypersensitivity of the glans penis frequently leads to the onset of ejaculation before or shortly after vaginal penetration, or an inability to keep erection or control ejaculation for a sufficient amount of time for a partner's sexual pleasure. Hypersensitivity leading to rapid ejaculation and premature ejaculation represent debilitating sexual dysfunctions that can lead to an inability to enter into, or sustain, relationships, cause psychological damage to sufferers, and also impair reproductive success.

Treatments for hypersensitivity to sexual stimulation and premature ejaculation include psychological therapies, topical anesthetics, and the use of devices. All of these treatments have significant drawbacks. Psychological therapies benefit only a subset of patients and require specialized therapists who may not be available to all patients. Furthermore, psychological therapies cannot alleviate pathologies resulting from non-psychological causes. Anesthetic agents decrease sensitivity of tissues, thereby diminishing sexual pleasure. Also, topical anesthetics can be transferred to sexual partners, thereby decreasing their sensitivity and pleasure as well. With regard to devices, these can be awkward, inconvenient and embarrassing to use. Devices are highly conspicuous and reveal the very condition that the suffering partner may prefer to conceal. Additionally, devices can cause irritation to one or both partners. The systemic administration of some antidepressant compounds (including fluoxetine, sertraline, paroxetine) have been described. See U.S. Pat. Nos. 4,507,323, 4,940,731, 5,151,448, and 5,276,042 and Rosen et al., J. Clin. Psychopharmacol., 19, 67-85 (1999) has also been tried in treating rapid and premature ejaculation. However, these antidepressants may not be effective for all patients, and their side effects can halt treatment or impair patient compliance. Disease states or adverse interactions with other drugs may contraindicate the use of these compounds or require lower dosages that may not be effective to delay the onset of ejaculation.

Thus, a need clearly exists for methods of delaying ejaculation that require no specialized psychological therapy, can be used conveniently and without embarrassment, and does not involve the problems associated with prior therapeutic methods. Specifically, there is a need for treatments of hypersensitivity of the glans penis that do not require the use of cumbersome devices, systemic administration of drugs with frequent, drug-limiting side effects or topical agents that adversely effect the sensitivity and sexual experience of the person's partner.

### SUMMARY OF THE DISCLOSURE

The present disclosure is drawn to a topical composition that effectively lowers the sensitivity of the glans penis without transferring the effects of decreased sensitivity to a person's partner following application thereby delaying ejaculation without embarrassment or systemic side effects or the need for ineffective psychological counseling.

Embodiments of the disclosure address the above-described need in the art by providing a novel method for treating hypersensitivity of sexual response and rapid ejaculation and premature ejaculation by locally administering an effective amount of a selected pharmacologically active agent to an individual in need of such therapy.

An embodiment of the disclosure includes a method wherein the pharmacologically active agent is administered topically to the penis. More particularly, an embodiment of the disclosure provides such a method wherein the pharmacologically active agent is administered topically to the glans penis.

An embodiment of the disclosure includes a method wherein the pharmacologically active agent effectively decreases the mean latency of dorsal nerve and glans penis somatosensory evoked potentials.

An embodiment of the disclosure includes a method wherein the pharmacologically active agent effectively increases the mean amplitude of dorsal nerve and glans penis somatosensory evoked potentials.

One embodiment of the disclosure includes a method wherein the pharmacologically active agent is a peptide.

An embodiment of the disclosure includes a method wherein the pharmacologically active agent is a topically active peptide enkephalin-stabilizing and/or enkephalin-releasing agent.

Embodiments of the disclosure also include pharmaceutical formulations for carrying out the aforementioned methods.

Further embodiments of the disclosure also include a kit capable of use by an individual in carrying out the aforementioned methods.

Additional objects, advantages and novel features of the disclosure will be set forth in part in the description which follows, and in part will become apparent to those skilled in the art upon examination of the following disclosure, or may be learned by practice of the disclosure.

One exemplary method in accordance with the disclosure is provided for treating male sexual hypersensitivity that may lead to rapid or premature ejaculation. This method comprises topically administering to an individual in need of such treatment an effective amount of a pharmaceutical formulation containing at least a vasodilator and a pharmacologically active desensitizing agent/drug. The pharmacologically active agent will generally, although not necessarily, be a peptide that may stimulate the release of an enkephalin and/or decrease the degradation of an enkephalin within the skin of the penis Administration of the pharmaceutical formulation is carried out within the context of a predetermined dosing regimen such that the agent is effective in the treatment of rapid or premature ejaculation. Agent delivery is preferably effected using topical or transdermal administration.

Surprisingly it has been found that the composition of the present disclosure may be administered without affecting a corresponding decrease in response in the female partner during intercourse. The preferred desensitizing agent is immediately absorbed through the skin of the penis and therefore does not need to subsequently be removed prior to intercourse. Partners of users have indicated no desensitization during intercourse.

A pharmaceutical formulation suitable for topical administration in the method of the disclosure comprises an effective amount of a selected pharmacologically active agent, a vasodilating agent, a carrier or vehicle suitable for local drug delivery, and, optionally, an enhancer. Other types of components may be incorporated into the formulation as well, e.g., excipients, surfactants, preservatives (e.g., antioxidants), stabilizers, enzyme inhibitors, chelating agents, and the like, as well as the components required to form a bio-compatible, pharmaceutically-acceptable, topical vehicle that is preferably water soluble, as will be appreciated by those skilled in the art of pharmaceutical formulation preparation and drug delivery.

A kit is also disclosed that provides an individual with the topical formulation and instructions for administering the formulation to treat rapid or premature ejaculation. Generally, the kit will include the following components: a pharmaceutical formulation comprising the pharmacologically active agent to be administered; a device for effecting delivery of the pharmaceutical formulation; a container housing the pharmaceutical formulation during storage and prior to use; and instructions for carrying out drug administration in a manner effective to delay the onset of ejaculation in a male individual.

### DETAILED DESCRIPTION OF THE DISCLOSURE

Before describing the details of the present disclosure, it is to be understood that this disclosure is not limited to one particular topical formulation or drug delivery system, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, for example, reference to “a pharmacologically active agent” includes a combination of two or more pharmacologically active agents, and the like.

As used herein, the transitional phrases “comprising”, “consisting essentially of and “consisting of” define the scope of the appended claims with respect to what un-recited additional components, if any, are excluded from the scope of the claim. The term “comprising” is intended to be inclusive or open-ended and does not exclude additional, un-recited elements or method steps. The phrase “consisting of excludes any element, step, or ingredient not specified in the claim. The phrase “consisting essentially of limits the scope of a claim to the specified materials or steps and those that do not materially affect the basic and novel characteristic(s) of the claimed disclosure. All compositions or formulations identified herein can, in alternate embodiments, be more specifically defined by any of the transitional phrases “comprising”, “consisting essentially of and “consisting of.”

In describing and claiming embodiments in the present disclosure, the following terminology will be used in accordance with the definitions set out below.

The terms “active agent,” “drug” and “pharmacologically active agent” are used interchangeably herein to refer to a chemical material or compound which, when administered to an organism (human or animal) induces a desired pharmacologic effect. In the preferred embodiment herein, the terms refer to a compound, which is capable of being administered topically. Included are derivatives and analogs of those compounds or classes of compounds that also induce the desired pharmacologic effect.

The term “topical administration” is used in its conventional sense to mean delivery of a topical drug or a pharmacologically active agent to the skin or mucosa.

“Carriers” or “vehicles” as used herein refer to carrier materials suitable for drug administration. Carriers and vehicles useful herein include any such materials known in the art, e.g., any liquid, gel, solvent, liquid diluent, solubilizer, or the like, which is nontoxic and which does not interact with other components of the composition in a deleterious manner.

The terms “rapid ejaculation” as used herein intends a sexual dysfunction wherein a male is unable to control the ejaculatory process to a degree sufficient to satisfy a partner. Generally, “premature ejaculation” refers to persistent or recurring ejaculation with limited stimulation before or during sexual intercourse. The term includes both “congenital” or “lifelong” premature ejaculation and “primary” or “acquired” premature ejaculation as set forth, for example, in U.S. Pat. No. 5,151,448, and in Male Infertility and Sexual Dysfunction at p. 356 (New York: Springer-Verlag, 1997). See also Diagnostic and Statistical Manual of Mental Disorders (Washington, D.C.: American Psychiatric Association, 1994).

By an “effective” amount of a drug or pharmacologically active agent is meant a nontoxic but sufficient amount of the drug or agent to provide a desired effect.

Active Agents for Delaying Ejaculation and/or Treating Rapid Ejaculation

In order to carry out the methods of the disclosure, a selected combination of one or more pharmacologically active agents is administered topically to the penis of a male individual. Suitable pharmacologically active agents include, but are not limited to Arginine, Niacin, Acetyl dipeptide-1 cetyl ester, and oligohexapeptides.

Arginine is an amino acid with vasodilatory properties. L-arginine is the primary nutrient that allows the body to create nitric oxide, which helps regulate every physiologic function in the body. L-arginine has been shown to reduce blood pressure, improve heart function, circulation, lower cholesterol, improve immune system response, wound healing, open airways in asthma and help with male sexual function. When arginine is introduced into the body, it interacts with the enzyme nitric oxide synthase, which replaces a nitrogen molecule on arginine with an oxygen atom forming nitric oxide. The compositions of the present disclosure may contain between about 10 mg and about 500 mg of arginine per 100 grams of the topical composition. Preferably, the compositions of the present disclosure contain between about 100 mg to about 300 mg of L-arginine per 100 grams of the topical composition. Most preferably, the compositions of the present disclosure contain about 250 mg of L-arginine per 100 grams of the topical composition. Arginine suitable for use in formulating compositions of the present disclosure is available commercially. For example, a commercial source of L-arginine that is suitable for use in compositions of the present disclosure is commercially available from T.J. Clark.

Niacin, also known as nicotinic acid or vitamin B3, is a water-soluble vitamin whose derivatives such as NADH, NAD, NAD+, and NADP play essential roles in energy metabolism in the living cell and DNA repair. The designation vitamin B3 also includes the corresponding amide nicotinamide (or “niacinamide”), whose chemical formula is C₆H₆N₂O. Other functions of niacin include removing toxic chemicals from the body, and assisting in the production of steroid hormones made by the adrenal gland, such as sex hormones and stress-related hormones. The compositions of the present disclosure may contain between about 10 mg to about 300 mg of niacin per 100 grams of the topical composition. Preferably, the compositions of the present disclosure contain between about 50 mg and about 200 mg of niacin per 100 grams of the topical composition. Most preferably, the compositions of the present disclosure contain about 100 mg of niacin per 100 grams of the topical composition. Niacin suitable for use in formulating compositions of the present disclosure is available commercially.

Acetyl dipeptide-1 cetyl ester, includes the dipeptide tyrosine-arginine and preferably, L-tyrosine-L-arginine, formulated as the N-acetyl salt of the hexadecyl ester of the dipeptide. The peptide is formulated this way to render the dipeptide lipophilic and effective on topical administration as is described in U.S. Pat. No. 6,372,717, which is incorporated herein by reference. This dipeptide, also known as kyotorphin, is a neuroactive peptide and neuromodulator known to play a role in pain regulation in the brain by enhancing release of a met-enkephalin and also by stabilizing the met-enkyphalin from degradation. It dampens or blocks nerve impulse generation. The compositions of the present disclosure may contain between about 0.10 mg to about 5 mg of acetyl dipeptide-1 cetyl ester per 100 grams of the topical composition. Preferably, the compositions of the present disclosure contain between about 0.5 mg and about 2 mg of acetyl dipeptide-1 cetyl ester per 100 grams of the topical composition. Most preferably, the compositions of the present disclosure contain about 1 mg of acetyl dipeptide-1 cetyl ester per 100 grams of the topical composition. Acetyl dipeptide-1 cetyl ester suitable for use in formulating compositions of the present disclosure is available commercially from Sederma, Inc, in Edison, N.J.

An oligohexapeptide is a peptide that is readily absorbed into the skin creating a lasting vasodilation that is chiefly responsible for enhancing erection quality and firmness when applied topically to the skin of the penis. Optionally, the compositions of the present disclosure may contain between about 0.1 mg to about 5 mg of oligohexapeptide per 100 grams of the topical composition. Most preferably, the compositions of the present disclosure contain about 1 mg of oligohexapeptide per 100 grams of the topical composition.

Preferably, the topical compositions of the present disclosure also contain a mixture of distilled water with at least one additional ingredient for formulating an pharmaceutically acceptable and elegant topical composition. Preferably, the compositions of the present disclosure are formulated in a topical lotion containing vitamin E. Another preferred embodiment is a condom having an internal surface coated with one of the topical compositions of the present disclosure.

The active agents may be administered in the form of pharmaceutically acceptable salts, esters, amides or prodrugs or combinations thereof. But conversion of inactive ester, amide or prodrug forms to an active form must occur prior to or upon reaching the target tissue or cell. Salts, esters, amides and prodrugs of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For example, acid addition salts are prepared from the free base (typically wherein the neutral form of the drug has a neutral NH₂ group) using conventional means, involving reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of acid moieties which may be present on a drug are prepared in a similar manner using a pharmaceutically acceptable base such as sodium hydroxide, potassium hydroxide, ammonium hydroxide, calcium hydroxide, trimethylamine, or the like. Preparation of esters involves functionalization of hydroxyl and/or carboxyl groups which may be present within the molecular structure of the drug. The esters are typically acyl-substituted derivatives of free alcohol groups, i.e., moieties which are derived from carboxylic acids of the formula RCOOH where R is alkyl, and preferably is lower alkyl. Esters can be reconverted to the free acids, if desired, by using conventional hydrogenolysis or hydrolysis procedures. Amides and prodrugs may also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety which results in a compound that is therapeutically inactive until modified by an individual's metabolic system.

### Pharmaceutical Formulations and Modes of Administration

The active agent or agents are preferably administered in a pharmaceutical formulation suitable for topical application to the male penis. The formulation preferably contains an effective amount of a vasodilator such as an oligohexapeptide, a desensitizing agent, preferably acetyl dipeptide-1 cetyl ester, and one or more selected carriers or excipients, such as water, as well as silicone, waxes, petroleum jelly, polyethylene glycol (“PEG”), propylene glycol (“PG”), liposomes, sugars such as mannitol and lactose, propylene glycol, di-PPG2-Myreth-10 Adipate, isopropyl myristate, butylene glycol, glycerin, glyceryl stearate, PEG-100 stearate, glycine soya (soy) bean oil, stearyl alcohol, dimethicone, hydrogenated lecithin, menthol, xanthan gum, lactic acid, tocopheryl acetate, laureth-3, hydroxyethylcellulose, phenoxyethanol, methylparaben, ethylparaben, propylparaben, butylparaben, isobutylparaben and/or a variety of other materials, having a pH of between about 5.0 and about 8.

The formulations may additionally include one or more enzyme inhibitors effective to inhibit drug-degrading enzymes which may be present in or on the skin. Such enzyme inhibiting compounds may be determined by those skilled in the art by reference to the pertinent literature and/or using routine experimental methods. Additional optional components include excipients, preservatives (e.g., antioxidants), chelating agents, solubilizing agents (e.g., surfactants), and the like, as will be appreciated by those skilled in the art of drug formulation preparation and delivery.

It may be desirable to deliver the active agent(s) in a dosage form which provides for controlled or sustained release of the agent(s). In such a case, the dosage form typically comprises a biocompatible, biodegradable material, typically a biodegradable polymer. Examples of such polymers include polyester, polyalkylcyanoacrylate, polyorthoester, polyanhydride, albumin, gelatin and starch. As explained, for example, in PCT Publication No. WO96/40054, these and other polymers can be used to provide biodegradable microparticles which enable controlled and sustained drug release, in turn minimizing the required dosing frequency.

The pharmaceutical formulations can be sterile or nonsterile and can either be manufactured under sterile conditions, thereby eliminating the need for post-manufacturing sterilization, or they can be manufactured under non-sterile conditions and optionally then subsequently sterilized by any suitable technique, e.g., radiation sterilization.

Preferred formulations for topical agent/drug delivery are ointments and creams. Ointments are semisolid preparations which are typically based on petrolatum or other petroleum derivatives. Creams containing the selected active agent, are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

The amount of active agents administered, and the dosing regimen used, will, of course, be dependent on the age and general condition and judgment of the male individual using the composition. Generally, the dosage will be administered locally just prior to intercourse. A typical dosage of the composition as administered locally is generally in the range of approximately one eighth of an ounce. This equates to about a quarter (25 cents) sized dab of the topical composition being applied directly to the penis. Depending on the response of the male individual, the dosing regimen can be modulated in order to achieve satisfactory control of the onset of ejaculation. By administering the drug locally, the side effects, drug interactions and disease considerations of systemic (e.g., oral) drug administration are avoided, as is the stigma associated with psychotherapeutic drug therapy.

Surprisingly it has been found and repeatedly demonstrated, through application of the topical formulations as above described and shown in detail in Table 1 below, by a number of male individuals immediately prior to intercourse, that their partners experience and report no loss of sensation during intercourse, even though desensitization of the penis is generally immediate. Thus ejaculation is effectively delayed without negatively affecting the sensory response in the partner. It is theorized that absorption of the vasodilator and desensitizing agent acetyl dipeptide-1 cetyl ester occurs virtually immediately upon application to the skin of the penis, and thus limited or no desensitizing agent remains on the surface of the individual's penis to be absorbed by vaginal tissues of the individual's partner. This dramatic result is a substantial benefit to both individuals involved.

### Kits

This disclosure also encompasses a kit for male individuals to carry out the present method of achieving delayed ejaculation using local drug therapy. The kit in accordance with the present disclosure contains the pharmaceutical formulation incorporating a vasodilator and desensitizing agent to be administered, a container, preferably sealed, for housing the composition during storage and prior to use, and instructions for carrying out administration of the composition in an effective manner. The instructions are preferably in written or pictograph form. However, such instructions may alternatively be provided on prerecorded media including audio tape, video tape, or the like

It is to be understood that while the disclosure has been described in conjunction with the preferred specific embodiments thereof, that the foregoing description as well as the examples which follow are intended to illustrate and not limit the scope of the disclosure.

### EXAMPLES

Fifteen different formulations were prepared and evaluated. The topical formulations contained the following active ingredients:

Formulation 1: arginine, niacin, acetyl dipeptide-1-cetyl ester, oligohexapaptide, menthol.

Formulation 2: arginine, niacin, acetyl dipeptide-1-cetyl ester, oligohexapaptide.

Formulation 3: arginine, niacin, acetyl dipeptide-1-cetyl ester, menthol.

Formulation 4: arginine, niacin, oligohexapaptide, menthol.

Formulation 5: arginine, acetyl dipeptide-1-cetyl ester, oligohexapaptide, menthol.

Formulation 6: niacin, acetyl dipeptide-1-cetyl ester, oligohexapaptide, menthol.

Formulation 7: arginine, niacin, acetyl dipeptide-1-cetyl ester.

Formulation 8: arginine, acetyl dipeptide-1-cetyl ester.

Formulation 9: arginine, acetyl dipeptide-1-cetyl ester, oligohexapaptide.

Formulation 10: acetyl dipeptide-1-cetyl ester, oligohexapaptide.

Formulation 11: niacin, oligohexapaptide.

Formulation 12: arginine, oligohexapaptide.

Formulation 13: niacin, acetyl dipeptide-1-cetyl ester, oligohexapaptide.

Formulation 14: niacin, arginine.

Formulation 15: acetyl dipeptide-1-cetyl ester, menthol.

For each of the above formulations, the active ingredient(s) were formulated as a pharmaceutical composition with at least one of the following ingredients to prepare a suitable topical formulation: propylene glycol, di-PPG2-Myreth-10 Adipate, isopropyl myristate, butylene glycol, glycerin, glyceryl stearate, PEG-100 stearate, glycine soya (soy) bean oil, stearyl alcohol, dimethicone, hydrogenated lecithin, menthol, xanthan gum, lactic acid, tocopheryl acetate, laureth-3, hydroxyethylcellulose, phenoxyethanol, methylparaben, ethylparaben, propylparaben, butylparaben, and isobutylparaben.

Of these formulations, Formulation 1 has been found to be particularly effective and desirable.

In particular, details of Formulation 1, which as been found to be effective for delaying ejaculation and/or treating rapid ejaculation or premature ejaculation was formulated for topical application to the glans penis, contains the ingredients set forth in Table 1 below.

TABLE 1
Topical Composition A

| INGREDIENT | gram weight % |
|---|---|
| Water | QS |
| Propylene Glycol | 15.00 |
| Di-PPG2-Myreth-10 Adipate | 6.00 |
| Isopropyl Myristate | 5.00 |
| Butylene Glycol | 4.00 |
| Glycerin | 4.00 |
| Glyceryl Stearate | 3.00 |
| PEG-100 Stearate | 2.00 |
| Glycine Soya (Soy) Bean Oil | 2.00 |
| Stearyl Alcohol | 2.00 |
| Dimethicone | 0.50 |
| Hydrogenated Lecithin | 0.50 |
| Menthol | 0.20 |
| Xanthan Gum | 0.25 |
| Arginine | 0.25 |
| Lactic Acid | 0.10 |
| Niacin | 0.10 |
| Tocopheryl Acetate | 0.10 |
| Laureth-3 | 0.10 |
| Hydroxyethylcellulose | 0.03 |
| Acetyl Dipeptide-1 Cetyl Ester | 0.001 |
| Oligohexapeptide | 0.001 |
| Phenoxyethanol | 0.72 |
| Methylparaben | 0.16 |
| Ethylparaben | 0.04 |
| Propylparaben | 0.02 |
| Butylparaben | 0.04 |
| Isobutylparaben | 0.02 |

Another composition that is believed to be effective is set forth in Table 2 below.

TABLE 1
Topical Composition B

| INGREDIENT | Gram Weight % |
|---|---|
| Water | 72.123 |
| Propylene Glycol | 8.000 |
| Cyclopentasiloxane | 3.500 |
| Di-PPG2-Myreth-10 Adipate | 3.500 |
| Isopropyl Myristate | 5.000 |
| Butylene Glycol | 1.500 |
| Glycerin | 1.750 |
| Glyceryl Stearate | 1.500 |
| PEG-100 Stearate | 1.500 |
| Glycine Soya (Soy) Bean Oil | 1.000 |
| Cetearyl Alcohol | 0.770 |
| Phenoxyethanol | 0.720 |
| Dimethicone | 0.500 |
| Hydrogenated Lecithin | 0.500 |
| Menthol | 0.200 |
| Xanthan Gum | 0.300 |
| Arginine | 0.250 |
| Ceteareth-20 | 0.230 |
| Acrylamide/Sodium Acryloylmethyltaurate copolymer | 0.125 |
| Lactic Acid | 0.100 |
| Niacin | 0.100 |
| Tocopheryl Acetate | 0.100 |
| Laureth-7 | 0.110 |
| Laureth-3 | 0.100 |
| Hydroxyethylcellulose | 0.050 |
| Acetyl Dipeptide-1 Cetyl Ester | 0.001 |
| Oligohexapeptide | 0.001 |
| C13-14 Isoparaben | 0.040 |
| Propylparaben | 0.020 |
| Ethylparaben | 0.040 |
| Isobtylparaben | 0.020 |
| Butylparaben | 0.040 |

Each of these compositions A and B contains a combination of vasodilators, including an oligohexapeptide, and the desensitizing agent acetyl dipeptide-1 cetyl ester.

The foregoing description of the present disclosure has been presented for purposes of illustration and description. Furthermore, the description is not intended to limit the disclosure to the form disclosed herein. Consequently, variations and modifications commensurate with the above teachings, and the skill or knowledge of the relevant art, are within the scope of the present disclosure. The embodiments described hereinabove are further intended to explain the best mode known for practicing the disclosure and to enable others skilled in the art to utilize the disclosure in such, or other, embodiments and with various modifications required by the particular applications or uses of the present disclosure. It is intended that the appended claims be construed to include alternative embodiments to the extent permitted by the prior art.

What is claimed is:

1. A method for treating a sexual dysfunction of premature ejaculation in a male patient in need thereof comprising topically administering to the penis or glans penis of the patient prior to sexual intercourse a therapeutically effective amount of a composition comprising at least one vasodilator agent, at least one desensitizing agent, and a pharmaceutically acceptable carrier.

2. The method according to claim 1 wherein the at least one vasodilator agent is an oligohexapeptide.

3. The method according to claim 1 wherein the at least one desensitizing agent is an acetyl dipeptide-1 cetyl ester.

4. The method according to claim 3 wherein the at least one vasodilator agent is an oligohexapeptide.

5. A method of delaying ejaculation during sexual intercourse between a male partner and a female partner without adversely affecting response in the female partner, the method comprising:
applying, prior to sexual intercourse, to the glans penis of the male partner, a therapeutically effective amount of a composition comprising at least one vasodilator agent, at least one desensitizing agent, and a pharmaceutically acceptable carrier.

6. The method according to claim 5 wherein the at least one vasodilator agent is an oligohexapeptide.

7. The method according to claim 5 wherein the at least one desensitizing agent is an acetyl dipeptide-1 cetyl ester.

8. The method according to claim 7 wherein the at least one vasodilator agent is an oligohexapeptide.

9. The method according to claim 5 wherein the composition is applied immediately prior to sexual intercourse.

10. A kit for use by a male patient to delay ejaculation during sexual intercourse, the kit comprising:
a container;
a composition within the container comprising a therapeutically effective amount of a composition comprising at least one vasodilator agent, at least one desensitizing agent, and a pharmaceutically acceptable carrier; and
a set of instructions for applying the composition to a male patient's penis prior to sexual intercourse.

11. The kit according to claim 10 wherein the vasodilator agent comprises an oligohexapeptide.

12. The kit according to claim 10 wherein the desensitizing agent comprise an acetyl dipeptide-1 cetyl ester.

13. The kit according to claim 12 wherein the vasodilator agent comprises an oligohexapeptide.

14. A topical composition for the treatment of premature ejaculation in a male patient without affecting a response in a partner during sexual intercourse, the topical composition comprising:
a vasodilator agent, and desensitizing agent and a pharmaceutically acceptable carrier.

15. The topical composition of claim 14 wherein the vasodilator agent is an oligohexapeptide and the desensitizing agent is an acetyl dipeptide-1 cetyl ester.

16. The topical composition of claim 15 further comprising propylene glycol and menthol.

17. The topical composition of claim 15 further comprising arginine.

18. A topical composition for the treatment of premature ejaculation in a male patient without adversely affecting response in a partner during sexual intercourse, the topical composition comprising:
acetyl dipeptide-1 cetyl ester, oligohexapeptide, arginine, menthol, water, propylene glycol, and niacin.
